(19) United States
(12) Patent Application Publication
(10) Pub. No.: US 2024/0165019 A1
(43) Pub. Date: May 23, 2024

(54) Methods for Treating Eyetear Film Deficiency

(71) Applicant: Bausch + Lomb Ireland Limited, Dublin (IE)

(72) Inventor: Annabelle Gallois-Bernos, Jacksonville, FL (US)

(21) Appl. No.: 17/991,113

(22) Filed: Nov. 21, 2022

(51) Int. Cl. A61K 9/00; A61K 31/728; A61P 27/04

(52) U.S. Cl. CPC A61K 9/0051; A61K 31/728; A61P 27/04

(57) ABSTRACT

The present invention relates to methods for treating tear film deficiencies in the eye by applying to the eye or the surrounding area a composition comprising hyaluronic acid.

### FIELD OF TECHNOLOGY

The present invention relates to methods for treating tear film deficiencies in the eye by applying to the eye or the surrounding area a composition comprising hyaluronic acid.

### BACKGROUND OF THE INVENTION

Dry eye is a debilitating condition causing dry-eye symptoms such as discomfort feelings on eyes and abnormalities in visual performance, associated with abnormalities in tear volume or quality, which may cause disorders on the surface of eyes such as cornea. The abnormalities in tear volume mainly refer to the condition of low lacrimal secretion, which is an index of dry eye based on the tear volume. Regarding the abnormalities in tear quality, abnormalities in tear ingredients such as few lipid or protein components contained in tears may deteriorate the stability of tear film, which may then cause dryness on the surface of eyes even with tear secretion.

When dry eye is associated with deficiencies or abnormalities in tear (or tear film) quality, dry eye may be evaporative dry eye, and may be associated with meibomian gland dysfunction (MGD). MGD results from obstruction of meibomian glands and is a major cause of dry eye. Tear film stability is one of the ocular surface homeostatic signs and is key to preventing the desiccating stress that cause or perpetuate dry eye disease and that lead to compromise of ocular surface health.

Hyaluronic acid occurs naturally in the human body and has been shown to effectively treat symptoms of dry eye. Though long-term studies have yet to be done, treatment with sodium hyaluronate appears to accelerate recovery of the damaged cornea (Katsuyama I, Arakawa T. A convenient rabbit model of ocular epithelium damage induced by osmotic dehydration. J Ocul Pharmacol Tuer 2003 June; 19 (3): 281-9). Sodium hyaluronate eye drops increase precorneal tear film stability and corneal wettability, reduce the tear evaporation rate, and the healing time of corneal epithelium (Aragona P, Di Stefano G, Ferreri F, et al. Sodium hyaluronate eye drops of different osmolarity for the treatment of dry eye in Sjogren's syndrome patients. Br J Ophthalmol 2002 August; 86 (8): 879-84). Sodium hyaluronate can be found in AQuify contact lens comfort drops (CIBA Vision).

### DESCRIPTION OF FIGURES

FIG. 1 shows results of MGD Rabbit Model for Determining Effect of Composition of Table 1 on NIBUT for Rabbit Groups 1 and 2 Measurements (average 3 measurements performed on healthy eye (control OS), Control OD (meibomian gland obstructed eye) and treated eye receiving Composition of Table 1 for Days 15-18).

### SUMMARY OF THE INVENTION

The present invention relates to methods of treating, preventing or reducing the symptoms associated with tear film deficiency in one or both eyes of a subject in need thereof, comprising the step of administering a composition comprising a hyaluronic acid compound to the eye(s), lacrimal gland(s) and/or the meibomian gland(s) of the subject.

The present invention also relates to methods of increasing lacrimal secretion in one or both eyes of a subject in need thereof, comprising the step of administering a composition comprising a hyaluronic acid compound to the eye(s) and/or lacrimal gland(s) of the subject.

The present invention also relates to methods of increasing meibomian gland secretion and/or meibomian gland in one or both eyes of a subject in need thereof, comprising the step of administering a composition comprising a hyaluronic acid compound to the eye(s) and/or the meibomian gland(s) of the subject

The present invention also relates to methods of increasing tear film breakup time in one or both eyes of a subject in need thereof, comprising the step of administering a composition comprising a hyaluronic acid compound to the eye(s), lacrimal gland(s) and/or the meibomian gland(s) of the subject

The present invention also relates to methods of preventing, reducing and/or treating meibomian gland dysfunction in one or both eyes of a subject in need thereof, comprising the step of administering a composition comprising a hyaluronic acid compound to the eye(s) and/or the meibomian gland(s) of the subject.

The present invention further relates to methods of treating, reducing, relieving or preventing evaporative dry eye due to reduced and/or the inhibition or blockage of meibomian gland secretions in one or both eyes of a subject in need thereof, comprising the step of administering a comprising a hyaluronic acid compound to the eye(s), lacrimal gland(s) and/or the meibomian gland(s) of the subject.

The present invention still further relates to methods of treating, reducing, relieving or preventing low, decreased or decreasing tear film break up times in one or both eyes of a subject in need thereof due to meibomian gland dysfunction, comprising the step of administering a composition comprising a hyaluronic acid compound to the eye(s), lacrimal gland(s) and/or the meibomian gland(s) of the subject.

The ophthalmic solutions may be dispensed to the eye of a subject up to four times or more per day per eye, as needed, to of reducing, relieving or preventing dry eye symptoms due to meibomian gland disfunction.

### DETAILED DESCRIPTION OF THE INVENTION

The methods of the invention are directed to ophthalmic solutions or, optionally, emulsions which contain a water-soluble polymer such as hyaluronic acid.

Additional utilities or benefits provided by the methods of the present invention include, without limitation, treating tear film instability, enhancing lacrimal and/or meibomian gland secretion, increasing tear volume, or the prevention, reduction or treatment of meibomian gland dysfunction, enhanced contact lens cleaning, prevention of contact lens water loss, inhibition of protein deposition on contact lenses and the like.

The methods and compositions of the present invention can comprise, consist of, or consist essentially of the essential elements and limitations of the invention described herein, as well any of the additional or optional features, components, or limitations described herein.

The term “comprising” (and its grammatical variations) as used herein is used in the inclusive sense of (and, interchangeably with the terms) “having” or “including” and not in the exclusive sense of “consisting only of.”

The terms “a” and “the” as used herein are understood to encompass the plural as well as the singular. Also, as used herein, “a,” “an,” “the,” “at least one,” and “one or more” are used interchangeably. Thus, for example, a composition that comprises “an” element can be interpreted to mean that the composition includes “one or more” such elements.

All percentages, parts and ratios are based upon the total weight of the composition of the present invention, unless otherwise specified.

All such weights as they pertain to the listed ingredients provided by way of example are based on the active level and, therefore, do not include carriers or by-products that may be included in commercially available materials, unless otherwise specified.

Unless otherwise indicated, all documents cited are, in relevant part, incorporated herein by reference; the citation of any document is not to be construed as an admission that it is prior art with response to the present invention. Furthermore, all documents incorporated herein by reference are only incorporated herein to the extent that they are not inconsistent with this specification.

In certain embodiments, the present invention as disclosed herein may be practiced in the absence of any compound, method step, or element (or group of compounds or elements) which is not specifically disclosed herein.

Unless defined otherwise, all technical and scientific terms used herein have the same meaning as commonly understood by one of ordinary skill in the art to which the invention belongs.

The methods of the invention are directed at preventing, reducing or relieving, or treating evaporative dry eye (symptoms thereof) due to meibomian gland disfunction (i.e., reduced or no secretions from one or more meibomian glands or poor quality secretions, resulting in tear film deficiency) comprising, consisting of, or consisting essentially of administering a safe and effective amount of an ophthalmic composition comprising, consisting of, or consisting essentially of a hyaluronic acid compound. Also disclosed herein are methods of preventing, reducing or relieving, or treating tear film instability, enhancing lacrimal and/or meibomian gland secretion, increasing tear volume, or the prevention, reduction or treatment of meibomian gland dysfunction.

The term “stable” is used in its customary sense and means the absence of creaming, flocculation, and phase separation.

The term “demulcent” is used in the usual sense and refers to an agent that relieves irritation of inflamed or abraded lens and/or eye surfaces.

The term “re-wetting” as used herein refers to the addition of liquid over at least a part, for example, at least a substantial part, of at least the anterior surface of a contact lens.

The term “tear film deficiency”, as used herein, means abnormalities in the tear film resulting in insufficient supply and/or excessive loss, and anomalous tear composition which reduce tear film stability or result in tear film instability. Such deficiency is typically caused by decreased (from typical amounts of) lacrimal and/or meibomian gland secretion and/or decreased (from typical amounts of) tear volume. Such decrease in lacrimal and/or meibomian gland secretion and/or decrease in tear volume can result in tear film breakup times of lower than 10 seconds—indicative of tear film instability. Tear film breakup times of 10 to 35 seconds are considered normal. (See Khurana, A K. “Diseases of lacrimal apparatus”. Comprehensive ophthalmology (6th ed.). Jaypee, The Health Sciences Publisher. p. 389. ISBN 978-93-5152-657-5.

The terms “treat” and “treatment” refer to the treatment of a patient afflicted with a pathological condition and refers to an effect that alleviates the condition by improving the stability or quality of the tear film, such as the thickness or balance of tear ingredients such as lipid or protein components and combinations of the foregoing. Treat and treatment also refer to an effect that results in the inhibition of the progress of the condition, and includes a reduction in the rate of progress, a halt in the rate of progress, amelioration of the condition. Treatment as a prophylactic measure (i.e., prophylaxis) is also included.

The term “meibomian gland disfunction” means obstruction, blockage or loss of function of one or more meibomian glands. Meimbomian gland disfunction may be observed by at least one sign or symptom including corneal staining, ocular dryness, itching, burning, redness, conjunctivitis, light sensitivity, ocular discharge, thick meibum, a reduced quantity of meibum on the ocular surface. Ocular dryness may be characterized by measuring the tear film break up time as described herein. Meibomian gland disfunction is a leading cause of evaporative dry eye and can worsen over time if left untreated. Evaporative dry eye is due to a deficient tear film lipid layer, which increases tear evaporation. It is caused by meibomian gland dysfunction, which occurs in over 85% of dry eye disease. (See Findlay Q, Reid K. Dry Eye Disease: When to Treat and When to Refer. Aust Prescr. 2018 October; 41(5):160-163.)

The term “ophthalmically compatible” means that the compound, component or solution does not have any substantial or significant detrimental effect on the (a) eye or ocular structure including the lids, ocular glands of the humans or animals to whom such compositions are administered, or (b) a contact lens being contacted or treated by the compositions of the methods of the present invention.

The term “safe and effective amount” refers to an amount of an active ingredient that elicits the desired biological or medicinal response in a subject's biological system without the risks outweighing the benefits of such response in accordance with the Federal Food, Drug, and Cosmetic Act, as amended (secs. 201-902, 52 Stat. 1040 et seq., as amended; 21 U.S.C. §§ 321-392). Safety is often measured by toxicity testing to determine the highest tolerable dose or the optimal dose of an active pharmaceutical ingredient needed to achieve the desired benefit. Studies that look at safety also seek to identify any potential adverse effects that may result from exposure to the drug. Efficacy is often measured by determining whether an active ingredient demonstrates a health benefit over a placebo or other intervention when tested in an appropriate situation, such as a controlled clinical trial or animal model testing.

The term “pharmaceutically acceptable” as used herein means being approved by a regulatory agency of the Federal or a state government, or listed in the U.S. Pharmacopeia, European Pharmacopeia or other generally recognized Pharmacopeia for use in animals, and more particularly in humans.

“Carriers” as used herein include carriers, excipients, or stabilizers that are nontoxic to the cell or mammal being exposed thereto at the dosages and concentrations employed. Often the carrier is an aqueous solution such as saline (e.g., 0.9% NaCl) or an aqueous pH buffered solution such as phosphate buffered saline (e.g., PBS).

The term “acceptable” with respect to a formulation, composition, or ingredient, as used herein, means that the beneficial effects of that formulation, composition, or ingredient on the general health of the patient being treated substantially outweigh its detrimental effects, to the extent any exist.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

The term “ophthalmic device” refers to an object that resides in or on the eye. These devices can provide optical correction or may be cosmetic. Ophthalmic devices include (selected from or selected from the group consisting of), but are not limited to, soft contact lenses, intraocular lenses, overlay lenses, ocular inserts, punctual plugs, and optical inserts.

### Hyaluronic Acid

The compositions used in the methods of the present invention comprise hyaluronic acid compounds (HA). Such compounds are natural polymers which can act as a demulcent in eye care compositions. As used herein, the term “hyaluronic acid compound (HA)” means hyaluronic acid and any of its hyaluronate salts, including, for example, sodium hyaluronate (the sodium salt), potassium hyaluronate, magnesium hyaluronate, and calcium hyaluronate.

HA is a polymer consisting of simple, repeating disaccharide units (glucuronic acid and N-acetyl glycosamine). It is made by connective tissue cells of all animals, and is present in large amounts in such tissues as the vitreous humor of the eye, the synovial fluids of joints, and the roostercomb of chickens. One method of isolating HA is to process tissue such as roostercombs. HA isolated from natural sources can be obtained from commercial suppliers, such as Biomatrix, Anika Therapeutics, ICN, and Pharmacia.

Another method of producing HA is via fermentation of bacteria, such as streptococci. The bacteria are incubated in a sugar rich broth and excrete HA into the broth. HA is then isolated from the broth and impurities are removed. The molecular weight of HA produced via fermentation may be altered by the sugars placed in the fermentation broth. HA produced via fermentation can be obtained from companies such as Bayer, Genzyme, and Fidia.

In its natural form, HA has a molecular weight in the range of 5×10⁴ up to 1×10⁷ daltons. Its molecular weight may be reduced via a number of cutting processes such as exposure to acid, heat (e.g. autoclave, microwave, dry heat), or ultrasonic waves. HA is soluble in water and can form highly viscous aqueous solutions. In the compositions of the present invention, the molecular weight for the HA is 8×10⁴ up to 1×10⁶ daltons, or 1.44×10⁶ to 1.66×10⁶ dalton.

The HA can be present in the compositions of present method at concentrations either alone or in combination with a second demulcent at concentrations of at a concentration of at least 0.001% (or about 0.001) w/w of the total composition.

Typically, HA is present at a concentration of from 0.01% (or about 0.01%) w/w to 1% (or about 1%) w/w, or from 0.05% (or about 0.05%) w/w to 0.5% (or about 0.5%) w/w, or from 0.1% (or about 0.1%) w/w to 0.3% (or about 0.3%) w/w, based on weight of the total composition. In certain embodiments, HA can be present as higher concentrations. HA is present at 0.15% (or about 0.15%) w/w to 0.25% (or about 0.01%) w/w, based on the weight of the total composition.

Methods of preparing or making the compositions used in the methods of present invention are disclosed. The present compositions are relatively easily and straight forwardly prepared and are storage-stable, for example, having a shelf life at about room temperature of at least about 2 years or more. In addition, the compositions of the methods of the present invention are advantageously easily sterilized, for example, using sterilizing techniques such as filtration sterilization, and eliminate, or at least substantially reduce, the opportunity or risk for microbial growth if the compositions become contaminated by inclusion of at least one anti-microbial agent.

### An Ophthalmologically Acceptable Carrier

The compositions of the methods of the present invention comprise an ophthalmologically acceptable carrier. The ophthalmologically acceptable carrier may be water or an aqueous excipient solution. The term “aqueous” typically denotes a formulation wherein the excipient is at least about 50%, or at least about 75%, or at least about 90% and up to at least about 95% or about 99%, by weight, water. The compositions of the methods of the present invention may contain an oil or oily component (e.g., castor oil, flaxseed oil and the like or mixtures thereof). The water can be distilled water. The carrier can be free of C1-4 alcohols such as methanol, ethanol, propanol, isopropanol, butanol, and the like which can sting, irritate, or otherwise cause discomfort to the eye.

The water may be present in the ophthalmologically acceptable carrier at concentrations of from about 96% to about 99.9%, or from about 98% to about 99.5%, or from about 99.0% to about 99.5%, by weight of the total composition.

The ophthalmologically acceptable carrier may be present at concentrations of from about 96% to about 99.5%, or from about 98% to about 99.5%, or from about 98.5% to about 99.2%, by weight of the total composition.

The compositions may be sterile, namely such that the absence of microbial contaminants in the product prior to release or use are statistically demonstrated to the degree necessary for such products. The compositions may be selected to have no or substantially no detrimental, negative, harmful effect on the contact lens being therein or on the eye (or on the region around the eye).

In certain embodiments, the compositions of the methods of the present invention are substantially free of oil or oily components. By the term “substantially free” as used with respect to the oil or lipid compounds, means the present compositions contain less than 0.05%, or less than 0.025% or less than 0.01%, or less than 0.005%, of such oils or oily components, by weight, based on the total composition.

The compositions according to the methods of the present invention are physiologically compatible with the eye and ophthalmic devices. Specifically, the composition should be “ophthalmologically safe” for use with an ophthalmic device such as a contact lens, meaning that a contact lens treated with the solution is generally suitable and safe for direct placement on or direct application to the eye without rinsing, that is, the solution is safe and comfortable for ophthalmic devices, of any frequency of application, wetted with the solution, including contact lenses of any wear frequency. An ophthalmologically safe composition has a tonicity and pH that is compatible with the eye and includes materials, and amounts thereof, that are ophthalmologically compatible and non-cytotoxic according to ISO standards and U.S. Food & Drug Administration (FDA) regulations.

The compositions of the methods of the present invention may be adjusted with tonicity agents, to approximate the osmotic pressure of normal lacrimal fluids, which is equivalent to a 0.9 percent solution of sodium chloride. The compositions may be made substantially isotonic with physiological saline used alone or in combination with other tonicity agents such as dextrose, otherwise if simply blended with sterile water and made hypotonic or made hypertonic the ophthalmic devices such as contact lenses may lose their desirable optical parameters. Correspondingly, excess saline may result in the formation of a hypertonic composition, which will cause stinging, and eye irritation. The osmolality of the composition may be at least about 200 mOsm/kg to less than 500 mOsm/kg, or from about 200 to about 450 mOsm/kg, or from about 205 to about 380 mOsm/kg, or from about 210 to about 360 milliosmoles per kilogram (mOsm/kg), or from about 250 to about 350 mOsm/kg, or from about 270 to about 330 mOsm/kg. The ophthalmic compositions will generally be formulated as sterile aqueous compositions.

Examples of suitable tonicity adjusting agents include (selected from or selected from the group consisting of), but are not limited to, sodium, potassium, calcium, zinc and magnesium chloride, alkali metal halides, dextrose, and the like and mixtures thereof. These agents may be used individually in amounts ranging from about 0.01 to about 2.5% w/v, or from about 0.2 to about 1.5% w/v, based on the total composition.

The tonicity adjusting agent may be sodium chloride which can be incorporated at concentrations of from about 0.4 to about 0.9, or from about 0.4 to about 0.7, or from about 0.5% to about 0.6%, by weight of the total composition.

The ophthalmologically acceptable carrier can contain one or more of the above-mentioned tonicity Agents.

In certain embodiments, the compositions of the methods of the present invention may further include a buffer component which is present in an amount effective to maintain the pH of the composition (or aqueous component thereof) in the desired range.

In certain embodiments, the compositions of the methods of the present invention (or aqueous phase/component thereof) may have a pH which is compatible with the intended use and is often in the range of about 4 to about 10. A variety of conventional buffers may be employed, such as phosphate, borate, citrate, acetate, histidine, tris, bis-tris and the like and mixtures thereof. Borate buffers include boric acid and its salts, such as sodium or potassium borate. Potassium tetraborate or potassium metaborate, which produce boric acid or a salt of boric acid in solution, may also be employed. Hydrated salts such as sodium borate decahydrate can also be used. Phosphate buffers include phosphoric acid and its salts; for example, M₂HPO₄ and MH₂PO₄, wherein M is an alkali metal such as sodium and potassium. Hydrated salts can also be used. In one embodiment of the present invention, Na₂HPO₄. 7H₂O and NaH₂PO₄·H₂O are used as buffers. The term phosphate also includes compounds that produce phosphoric acid or a salt of phosphoric acid in solution. Additionally, organic counter-ions for the above buffers may also be employed. The concentration of buffer generally varies from about 0.01 to 2.5 w/v %, or varies from about 0.05 to about 0.5 w/v %, based on the total composition.

The type and amount of buffer are selected so that the formulation meets the functional performance criteria of the composition, such as surfactant and shelf-life stability, antimicrobial efficacy, buffer capacity and the like factors. The buffer is also selected to provide a pH, which is compatible with the eye and any contact lenses with which the composition is intended for use. Generally, a pH close to that of human tears, such as a pH of from about 7.45, is very useful, although a wider pH range of from about 6 to about 9, or from about 6.5 to about 8.5, or from about 6.8 to about 8.0, or from about 7.3 to about 7.7 is also acceptable. In one embodiment, the compositions of the methods of the present invention have a pH of about 7.0.

The compositions of the methods of the present invention may also include one or more surfactants. Surfactants suitable for use include, but are not limited to, ionic and nonionic surfactants (though nonionic surfactants are preferred), RLM 100, POE 20 cetylstearyl ethers such as Procol® CS20, poloxamer block copolymers (such as Pluronic® F68, and block copolymers such as poly(oxyethylene)-poly(oxybutylene) compounds set forth in US2008/0138310 (which publication is herein incorporated by reference). The poly(oxyethylene)-poly(oxybutylene) block copolymer may have the formula (EO)ₘ(BO)ₙ, wherein EO is oxyethylene and BO is oxybutylene, and wherein m is an integer having an average value of 10 to 1000 and n is an integer having an average value of 5 to 1000, as disclosed in U.S. Pat. No. 8,318,144; m may also be 10 and n may be 5. Surfactants such as polyethylene glycol (PEG) hydrogenated castor oil may also be used. Combinations of any of the above-mentioned surfactants are also useful herein.

Surfactants may be present at concentrations of from about 0.01 to about 3%, or from about 0.01 to about 1%, or from about 0.02 to about 0.5%, or from about 0.02 to about 0.1%, by weight of the total composition of the present invention. It should be appreciated that some of the components may perform more than one function, for example, some demulcents may also function as surfactants (e.g., PEO-PPO and PEO-PBO block copolymers).

In addition to the HA mentioned above, the compositions used in the methods of the present invention may also include compounds which act as either demulcents and/or viscosity modifying agents or components.

Compounds preferred for use as viscosity modifying agents include cellulose polymers, including hydroxypropyl methyl cellulose (HPMC), hydroxyethyl cellulose (HEC), ethyl hydroxyethyl cellulose, hydroxypropyl cellulose, methyl cellulose and carboxymethyl cellulose; carbomers (e.g. carbopol. RTM); polyvinyl alcohol; polyvinyl pyrrolidone; alginates; carrageenans; and guar, karaya, agarose, locust bean, tragacanth and xanthan gums. Such viscosity modifying components are employed, if at all, in an amount effective to provide a desired viscosity to the compositions.

Compounds preferred for use as a second demulcent (in addition to the HA) include carboxymethylcellulose, other cellulose polymers (e.g., carboxy methyl cellulose methylcellulose, hydroxyethylcellulose, hydroxypropylcellulose, hydroxypropyl methylcellulose (HPMC) and methyl ethyl cellulose), dextran 70, gelatin, polyethylene glycols (e.g., PEG 300 and PEG 400), polyvinyl alcohol, polyvinylpyrrolidone (PVP) and the like and mixtures thereof, may also be incorporated into the compositions used in the methods of the present invention.

In more preferred embodiments, demulcents may further include water soluble polymers such as polyvinylpyrrolidone (PVP), and hydroxypropyl methylcellulose (HPMC), polyethylene glycols (e.g., PEG 300 and PEG 400) and salts thereof and mixtures thereof.

The concentration of such second demulcents or viscosity modifiers will typically vary between about 0.01 to about 5% w/v, based on the total composition, although other concentrations of certain viscosity modifying components may be employed

In certain embodiments, the compositions of the methods of the present invention further include sequestering agents or components in order to, and in an amount effective to, bind metal ions, which, for example, might otherwise stabilize cell membranes of microorganisms and thus interfere with optimal disinfection activity. Alternatively, it is desirable in some instances to bind metal ions to prevent their interaction with other species in the compositions. Sequestering agents are included, if at all, in amounts effective to bind at least a portion, for example, at least a major portion of the metal ions present. Such sequestering components usually are present in amounts ranging from about 0.01 to about 0.2 w/v %. Examples of useful sequestering components include, without limitation ethylene-diaminetetraacetic acid (EDTA) and its potassium or sodium salts and low molecular weight organic acids such as citric and tartaric acids and their salts, e.g., sodium salts.

The components used in accordance with the present invention are selected to be effective in the compositions of the methods of the present invention and to have no substantial or significant deleterious effect, for example, on the compositions, on the use of the compositions, on the ophthalmic device being treated, on the user of the treated ophthalmic device, or on the human or animal in whose eye the composition is placed.

The ophthalmic device may be a contact lens. Contact lenses useful with the compositions can be manufactured employing various conventional techniques, to yield a shaped article having the desired posterior and anterior lens surfaces. Spincasting methods are disclosed in U.S. Pat. Nos. 3,408,429 and 3,660,545; static casting methods are disclosed in U.S. Pat. Nos. 4,113,224, 4,197,266, and 5,271,875, each of which are herein incorporated by reference. Contact lens polymer materials useful for manufacturing suitable contact lenses include (selected from or selected from the group consisting of), but are not limited to, acofilcon A, alofilcon A, alphafilcon A, amifilcon A, astifilcon A, atalafilcon A, balafilcon A, bisfilcon A, bufilcon A, comfilcon, crofilcon A, cyclofilcon A, darfilcon A, deltafilcon A, delefilcon, deltafilcon B, dimefilcon A, drooxifilcon A, epsifilcon A, esterifilcon A, etafilcon A, fanfilcon A, focofilcon A, galyfilcon A, genfilcon A, govafilcon A, hefilcon A, hefilcon B, hefilcon D, hilafilcon A, hilafilcon B, hioxifilcon B, hioxifilcon C, hixoifilcon A, hydrofilcon A, lenefilcon A, licryfilcon A, licryfilcon B, lidofilcon A, lidofilcon B, lotrafilcon A, lotrafilcon B, mafilcon A, mesifilcon A, methafilcon B, mipafilcon A, narafilcon A, narafilcon B, nelfilcon A, netrafilcon A, ocufilcon A, ocufilcon B, ocufilcon C, ocufilcon D, ocufilcon E, ofilcon A, omafilcon A, oxyfilcon A, pentafilcon A, perfilcon A, pevafilcon A, phemfilcon A, polymacon, riofilcon A, samfilcon A, senofilcon A, senofilcon C, serafilcon, silafilcon A, siloxyfilcon A, somofilcon A, stenfilcon A, tefilcon A, tetrafilcon A, trifilcon A, vasurfilcon, verofilcon, vifilcon, and xylofilcon A. Prefereably, the contact lenses are manufactured using polymer materials selected from (or selected from the group consisting of) comfilcon, etafilcon A, galyfilcon A, senofilcon A, nelfilcon A, hilafilcon, tetrafilcon A, vasurfilcon, vifilcon, and polymacon.

Conventional hydrogel contact lenses do not contain silicone containing components, and generally have higher water content, lower oxygen permeability and moduli than silicone hydrogels. Conventional hydrogels are prepared from monomeric mixtures predominantly containing hydrophilic monomers, such as 2-hydroxyethyl methacrylate (“HEMA”), N-vinyl pyrrolidone (“NVP”) or polyvinyl alcohols. U.S. Pat. Nos. 4,495,313, 4,889,664 and 5,039,459 disclose the formation of conventional hydrogels. Conventional hydrogels may be ionic or non-ionic and include (selected from or selected from the group consisting of) polymacon, etafilcon, nelfilcon, ocufilcon lenefilcon and the like. The oxygen permeability of these conventional hydrogel materials is typically below 20-30 barrers.

Silicon hydrogel formulations include (selected from or selected from the group consisting of) balafilcon samfilcon, lotrafilcon A and B, delfilcon, galyfilcon, senofilcon A, B and C, narafilcon, comfilcon, formofilcon, riofilcon, fanfilcon, stenfilcon, somofilcon, serafilcon, kalifilcon and the like. “Silicone hydrogels” refer to polymeric networks made from at least one hydrophilic component and at least one silicone-containing component. Silicone hydrogels may have moduli in the range of 60-200, 60-150 or 80-130 psi, water contents in the range of 20 to 60%. Examples of silicone hydrogels include acquafilcon, asmofilcon, balafilcon, comfilcon, delefilcon, enfilcon, fanfilcon, formofilcon, galyfilcon, lotrafilcon, lehfilcon, kalifilcon, narafilcon, riofilcon, samfilcon, senofilcon, somofilcon, and stenfilcon, including all of their variants, as well as silicone hydrogels as prepared in U.S. Pat. Nos. 4,659,782, 4,659,783, 5,244,981, 5,314,960, 5,331,067, 5,371,147, 5,998,498, 6,087,415, 5,760,100, 5,776,999, 5,789,461, 5,849,811, 5,965,631, 6,367,929, 6,822,016, 6,867,245, 6,943,203, 7,247,692, 7,249,848, 7,553,880, 7,666,921, 7,786,185, 7,956,131, 8,022,158, 8,273,802, 8,399,538, 8,470,906, 8,450,387, 8,487,058, 8,507,577, 8,637,621, 8,703,891, 8,937,110, 8,937,111, 8,940,812, 9,056,878, 9,057,821, 9,125,808, 9,140,825, 9,156,934, 9,170,349, 9,244,196, 9,244,197, 9,260,544, 9,297,928, 9,297,929 as well as WO 03/22321, WO 2008/061992, and US 2010/0048847. These patents are hereby incorporated by reference in their entireties.

The contact lens polymer material is preferably a silicone hydrogel polymer. The silicone hydrogel may be selected from (or selected from the group consisting of) acquafilcon, asmofilcon, balafilcon A, comfilcon, delefilcon, enfilcon, galyfilcon, lotrafilcon, senofilcon, samfilcon, somofilcon, stenfilcon, lehfilcon, kalifilcon, serafilcon, and unifilcon.

The present invention provides for methods of using HA containing compositions, such as the ophthalmic compositions described herein. In one embodiment, the present methods comprise administering a composition of the invention to an eye of a subject, for example, a human or an animal, in an amount and at conditions effective to provide a benefit to the eye such as tear film stabilization, enhanced lacrimal and/or meibomian gland secretion, increased tear volume or the prevention, reduction or treatment of meibomian gland dysfunction. In this embodiment, the compositions of the methods of the present invention may employ at least one a therapeutic component and the like, useful for treating a condition, for example, dry eye and/or one or more other conditions of the eye.

In one embodiment, the compositions used in the methods of the present invention include a second therapeutic agent in addition to the HA.

The compositions used in the methods of the present invention are useful, for example, as a carrier or vehicle, for the delivery of at least one additional therapeutic agent to or through the eye. Any suitable therapeutic component may be included in the present compositions provided that such therapeutic component is compatible with the remainder of the composition, does not unduly interfere with the functioning and properties of the remainder of the composition, is effective, for example, to provide a desired therapeutic effect, when delivered in the present composition and is effective when administered to or through the eye.

Optionally, an effective amount of a desired second therapeutic agent or component preferably is physically combined or mixed with the other components of a composition of the present invention to form a therapeutic component-containing composition within the scope of the present invention.

The type of second therapeutic agent or agents used will depend primarily on the therapeutic effect desired, for example, the disease or disorder or condition to be treated. These therapeutic agents or components include a broad array of drugs or substances currently, or prospectively, delivered to or through the eye in topical fashion or otherwise. Examples of useful additional therapeutic components which may be used in conjunction with a treatment for dry eye include, but not limited to:
(1) anti-infective and anti-microbial substances including quinolones, such as ofloxacin, ciprofloxacin, norfloxacin, gatifloxacin and the like; beta-lactam antibiotics, such as cefoxitin, n-formamidoyl-thienamycin, other thienamycin derivatives, tetracyclines, chloramphenicol, neomycin, carbenicillin, colistin, penicillin G, polymyxin B, vancomycin, cefazolin, cephaloridine, chibrorifamycin, gramicidin, bacitracin sulfonamides and the like; aminoglycoside antibiotics, such as gentamycin, kanamycin, amikacin, sisomicin, tobramycin and the like; naladixic acid and analogs thereof and the like; antimicrobial combinations, such as fluealanine/pentizidone and the like; nitrofurazones; and the like and mixtures thereof;
(2) anti-allergy agents, antihistaminics, anti-hypertensive agents and decongestants, such as pyrilamine, chlorpheniramine, phenylephrine hydrochloride, tetrahydrazoline hydrochloride, naphazoline hydrochloride, oxymetazoline, antazoline, and the like and mixtures thereof;
(3) anti-inflammatories, such as cortisone, hydrocortisone, hydrocortisone acetate, betamethansone, dexamethasone, dexamethasone sodium phosphate, prednisone, methylprednisolone, medrysone, fluorometholone, fluocortolone, prednisolone, prednisolone sodium phosphate, triamcinolone, sulindac, salts and corresponding sulfides thereof, and the like and mixtures thereof;
(4) non-steroid anti-inflammatory drug (NSAID) components, such as those which do or do not include a carboxylic (—COOH) group or moiety, or a carboxylic derived group or moiety; NSAID components which inhibit, either selectively or non-selectively, the cyclo-oxygenase enzyme, which has two (2) isoforms, referred to as COX-1 and COX-2; phenylalkoanoic acids, such as diclofenac, flurbiprofen, ketorolac, piroximcam, suprofen and the like; indoles such as indomethacin and the like; diarylpyrazoles, such as celecoxib and the like; pyrrolo pyrroles; and other agents that inhibit prostaglandin synthesis and the like and mixtures thereof;
(5) miotics and anticholinergics, such as echothiophate, pilocarpine, physostigmine salicylate, diisopropylfluorophosphate, epinephrine, dipivolyl epinephrine, neostigmine, echothiopate iodide, demecarium bromide, carbachol, methacholine, bethanechol, and the like and mixtures thereof;
(6) mydriatics, such as atropine, homatropine, scopolamine, hydroxyamphetamine, ephedrine, cocaine, tropicamide, phenylephrine, cyclopentolate, oxyphenonium, eucatropine, and the like and mixtures thereof;
(7) antiglaucoma drugs, for example, prostaglandins, such as those described in U.S. Pat. Nos. 6,395,787 and 6,294,563, which are herein incorporated by reference in their entirety, adrenergic agonists such as quinoxalines and quinoxaline derivatives, such as (2-imidozolin-2-ylamino)quinoxaline, 5-halide-6-(2-imidozolin-2-ylamino)quinoxaline, for example, 5-bromo-6-(2-imidozolin-2-ylamino)quinoxaline and brimonidine and its derivatives, such as those described in U.S. Pat. No. 6,294,563, which is herein incorporated by reference in its entirety and the like, timolol, especially as the maleate salt and R-timolol and timolol derivatives and a combination of timolol or R-timolol with pilocarpine and the like; epinephrine and epinephrine complex or prodrugs such as the bitartrate, borate, hydrochloride and dipivefrin derivatives and the like; hyperosmotic and dipivefrin derivatives and the like; betaxolol, hyperosmotic agents, such as glycerol, mannitol and urea and the like and mixtures thereof;
(8) antiparasitic compounds and/or anti-protozoal compounds, such as ivermectin; pyrimethamine, trisulfapyrimidine, clindamycin and corticosteroid preparations and the like and mixtures thereof;
(9) antiviral compounds, such as acyclovir, 5-iodo-2′-deoxyuridine (IDU), adenosine arabinoside (Ara-A), trifluorothymidine, interferon and interferon inducing agents, such as Poly I:C and the like and mixtures thereof;
(10) carbonic anhydrase inhibitors, such as acetazolamide, dichlorphenamide, 2-(p-hydroxyphenyl)thio-5-thiophenesulfonamide, 6-hydroxy-2-benzothiazole-sulfonamide 6-pivaloyloxy-2-benzothiazolesulfonamide and the like and mixtures thereof;
(11) anti-fungal agents, such as amphotericin B, nystatin, flucytosine, natamycin, and miconazole and the like and mixtures thereof;
(12) pain-relieving and anesthetic agents, such as etidocaine, cocaine, benoxinate, dibucaine dydrochloride, dyclonine hydrocholoride, naepaine, phenacaine hydrochloride, piperocaine, proparacaine hydrochloride, tetracaine hydrochloide, hexylcaine, bupivacaine, lidocaine, mepivacaine and prilocaine and the like and mixtures thereof;
(13) ophthalmic diagnostic agents, such as
(a) those used to examine the retina, such as chloride-sodium fluorescein and the like and mixtures thereof;
(b) those used to examine the conjunctiva, cornea and lacrimal structures, such as fluorescein and rose Bengal and the like and mixtures thereof; and
(c) those used to examine abnormal pupillary responses such as methacholine, cocaine, adrenaline, atropine, hydroxyamphetamine and pilocarpine and the like and mixtures thereof;
(14) ophthalmic agents used as adjuncts in surgery, such as alpha-chymotrypsin, and hyaluronidase and the like;
(15) chelating agents, such as ethylenediamine tetraacetate (EDTA) and deferoxamine and the like; and mixtures thereof;
(16) immunosuppressive agents and anti-metabolites, such as methotrexate, cyclophosphamide, 6-mercaptopurine, cyclosporins such A through I and azathioprine and the like; and mixtures thereof;
(17) angiostatic agents;
(18) muco-secretogogue agents;
(19) proteins and growth factors such as epidermal growth factor;
(20) vitamins and vitamin derivatives such as vitamins A, B12, C, D, E, folic acid and their derivatives;
(21) combinations of the above such as antibiotic/anti-inflammatory as in neomycin sulfate-dexamethasone sodium phosphate, quinolone-NSAID and the like; and concomitant anti-glaucoma therapy, such as timolol maleate-aceclidine and the like.

When a second therapeutic component is present in the compositions used in the methods of the present invention, the amount of such therapeutic component in the composition preferably is effective to provide the desired therapeutic effect to the human or animal to whom the composition is administered.

Typically, when a second therapeutic component is present, the compositions comprising oil-in-water emulsions of the present invention may contain from or at least about 0.001%, for example, about 0.01%, to about 5% (w/v) of the therapeutic component, e.g., medicament or pharmaceutical, on a weight to weight basis. Thus, for example, from one drop of liquid composition which contains about 25 mg of composition, one would obtain about 0.0025 mg to about 1.25 mg of therapeutic component.

The particular therapeutic component, e.g., drug or medicament, used in the pharmaceutical compositions of this invention is the type which a patient would require or benefit from for the treatment, e.g., pharmacological treatment, of a condition which the patient has or is to be protected from or from which the patient is suffering. For example, if the patient is suffering from glaucoma, the drug of choice may be timolol and/or one or more other anti-glaucoma components.

It is within the knowledge of one skilled in the art to determine the correct amounts of therapeutic component, e.g., drug, to be added to a composition of the invention in order to assure the efficacious delivery of the desired therapeutic component.

All types of contact lenses may be used in association with the methods of the present invention. For example, such contact lenses may be soft, rigid and soft or flexible gas permeable, silicone hydrogel, silicon non-hydrogel and conventional hard contact lenses.

The present compositions used in the methods of the present invention may further comprise antimicrobial agents (i.e., preservatives or disinfectants) to preserve the compositions from microbial contamination and/or disinfect contact lenses. The amount of the disinfectant component present in the liquid aqueous medium is effective to disinfect a contact lens placed in contact with the composition.

For example, the disinfectant component may include, but is not limited to, quaternary ammonium salts used in ophthalmic applications such as poly[dimethylimino-w-butene-1,4-diyl]chloride, alpha[4-tris(2-hydroxyethyl)ammonium]-dichloride (chemical registry number 75345-27-6, available under the trademark Polyquaternium 1® (from Onyx Corporation), poly(oxyethyl(dimethyliminio)ethylene dmethyliminio) ethylene dichloride sold under the trademark WSCP by Buckman laboratories, Inc. in Memphis, Tenn., benzalkonium halides, salts of alexidine, alexidine-free base, salts of chlorhexidine, hexetidine, alkylamines, alkyl di-and tri-amine, tromethamine (2-amino-2-hydroxymethyl-1, 3propanediol), hexamethylene biguanides and their polymers, cetylpyridinium chloride, cetylpyridinium salts, antimicrobial polypeptides, and the like and mixtures thereof. A particularly useful disinfectant component is selected from one or more (mixtures) of polyhexamethylene biguanide (PHMB), Polyquaternium-1, ophthalmically acceptable salts thereof, and the like and mixtures thereof.

The salts of alexidine and chlorhexidine can be either organic or inorganic and are typically disinfecting gluconates, nitrates, acetates, phosphates, sulphates, halides and the like. Generally, the hexamethylene biguanide polymers, also referred to as polyaminopropyl biguanide (PAPB), have molecular weights of up to about 100,000. Such compounds are known and are disclosed in U.S. Pat. No. 4,758,595 which is incorporated in its entirety by reference herein.

The disinfectant components useful in the present invention are present in the compositions of the present invention at concentrations in the range of about 0.00001% to about 2% (w/v).

The disinfectant component is present in the compositions of the present invention at an ophthalmically acceptable or safe concentration such that the user can remove the disinfected lens from the composition and thereafter directly place the lens in the eye for safe and comfortable wear.

When a contact lens is desired to be disinfected by a disinfectant component, an amount of disinfectant effective to disinfect the lens is used. An effective amount of the disinfectant reduces the microbial burden on the contact lens by one log order, in three hours. In preferred embodiments, an effective amount of the disinfectant reduces the microbial load by one log order in one hour.

The disinfectant component is preferably soluble in the compositions of the methods of the present invention (or aqueous component thereof).

The present compositions may include an effective amount of a preservative component. Any suitable preservative or combination of preservatives may be employed. Examples of suitable preservatives include, without limitation, chlorous acid compounds such as sodium chlorite or stabilized chlorine dioxide (SCD) Purogene® material (PUROGENE is a trademark of BioCide International, Inc. Norman, Okla., U.S.A., and is also available as Purite® which is a trademark of Allergan, Inc.), polyhexamethylene biguanide (PHMB), Polyquaternium-1, ophthalmically acceptable salts thereof, and the like and mixtures thereof, benzalkonium chloride, methyl and ethyl parabens, hexetidine and the like and mixtures thereof. The amount of preservative components included in the present compositions are such to be effective in preserving the compositions and can vary based on the specific preservative component employed, the specific composition involved, the specific application involved, and the like factors. Preservative concentrations often are in the range of about 0.00001% to about 0.05% or about 0.1% (w/v) of the composition, although other concentrations of certain preservatives may be employed.

Other useful preservatives include antimicrobial peptides. Among the antimicrobial peptides which may be employed include, without limitation, defensins, peptides related to defensins, cecropins, peptides related to cecropins, magainins and peptides related to magainins and other amino acid polymers with antibacterial, antifungal and/or antiviral activities. Mixtures of antimicrobial peptides or mixtures of antimicrobial peptides with other preservatives are also included within the scope of the present invention.

Preferred for use herein is a chlorous acid compound including (selected from or selected from the group consisting of), but not limited to, potassium chlorite, sodium chlorite, calcium chlorite, magnesium chlorite and mixtures thereof. In a preferred embodiment, the chlorite compound comprises sodium chlorite.

The present compositions may comprise effective amounts of one or more additional components. For example, one or more conditioning components or one or more contact lens wetting agents and the like and mixtures thereof may be included. Acceptable or effective concentrations for these and other additional components in the compositions of the invention are readily apparent to the skilled practitioner.

When any component is included, it is preferably compatible under typical use and storage conditions with the other components of the composition.

It will be understood by those of skill in the art that numerous and various modifications can be made without departing from the spirit of the present invention. Therefore, it should be clearly understood that the following examples are illustrative only and are not intended to limit the scope of the present invention.

Methods of Dosing and Treatment Regimes

In one aspect, described herein are methods of treating, reducing, relieving or preventing tear film instability in a mammal suffering from tear film instability in one or both eyes of the mammal by administering to at least one eye of said mammal a safe and effective amount of an ophthalmic solution comprising, consisting of, or consisting essentially of hyaluronic acid.

In one aspect, described herein are methods of treating, reducing, relieving or preventing evaporative dry eye due to meibomian gland disfunction in a mammal suffering from evaporative dry eye in one or both eyes of a subject in need thereof by administering to at least one eye of said mammal a safe and effective amount of an ophthalmic solution comprising, consisting of, or consisting essentially of hyaluronic acid

In one aspect, described herein are methods of treating, reducing, relieving or preventing decreased or completely inhibited lacrimal gland secretions due to obstruction or blockage of lacrimal glands in a mammal suffering from decreased or completely inhibited lacrimal gland secretion in one or both eyes of a subject in need thereof by administering to at least one eye of said mammal a safe and effective amount of an ophthalmic solution comprising, consisting of, or consisting essentially of hyaluronic acid.

In one aspect, described herein are methods of treating, reducing, relieving or preventing decreased or completely inhibited meibomian gland secretions due to obstruction or blockage of lacrimal glands in a mammal suffering from decreased or completely inhibited meibomian gland secretion in one or both eyes of a subject in need thereof by administering to at least one eye of said mammal a safe and effective amount of an ophthalmic solution comprising, consisting of, or consisting essentially of hyaluronic acid.

In one aspect, described herein are methods of treating, reducing, relieving or preventing low, decreased or decreasing tear film breakup time (i.e., Tear film breakup times at (or trending) below 15 seconds, below 10 seconds or below 5 seconds) due to lacrimal gland dysfunction and/or meibomian gland dysfunction in one or both eyes of a subject in need thereof by administering to at least one eye of said mammal a safe and effective amount of an ophthalmic solution comprising, consisting of, or consisting essentially of hyaluronic acid.

In one aspect, described herein are methods of treating, reducing, relieving or preventing meibomian gland dysfunction in one or both eyes of a subject in need thereof by administering to at least one eye of said mammal a safe and effective amount of an ophthalmic solution comprising, consisting of, or consisting essentially of hyaluronic acid.

The compositions may be administered to the eye(s) of the mammal suffering from meibomian gland dysfunction, lacrimal gland dysfunction, or tear film deficiency, as eye drops or indirectly through the use of contact lens soaked in the compositions of the present inventions. Contact lens care compositions include multipurpose cleaning, rinsing, disinfecting and storage compositions as well as rewetting, in-the-eye cleaning and other compositions for the contact lens. The compositions of the methods of the present invention may comprise additional components, for example other components for the treatment of dry eye (such as evaporative), or signs or symptoms of such dry eye, including dry eye symptoms due to meibomian gland disfunction, lacrimal gland dysfunction, or tear film deficiency.

The present inventor has found that by applying the certain compositions to the eyes and/or meibomian glands of mammals in accordance with the methods of present invention, the stability of tear films on the eyes can be significantly improved, as measured by non-invasive tear film breakup time. Specifically, the methods of the present invention can maintain (i.e., prevent further decrease in) the Non-Invasive Tear Break-Up Time (NITBUT) (a measure of tear film stability) so as to maintain the NITBUT within the range (e.g., within ±5% or ±3%) of NITBUT measurements obtained the day (or about the time—e.g., ±1 hour) of commencement of the methods of the present invention. If multiple such NITBUT measurements are taken in a day, the average of the NITBUT measurements is used as the “measurement obtained the day (or about the time—e.g., ±1 hour) of commencement of the methods of the present invention” for purposes of the present application; this measurement is considered the baseline for the NITBUT calculations—i.e., determining increase in seconds or percentage increase.

In certain embodiments, the methods of the present invention can increase the NITBUT beyond NITBUT measurement obtained the day (or about the time—e.g., ±1 hour) of commencement of the methods of the present invention by up to 1 (or about 1) seconds, up to 1.5 (or about 1.5) seconds or more, or by up to 2 (or about 2) seconds or more, after administration, which increase, in certain embodiments, can be observed within (or after) about 10 days or more, 20 days or more, or 30 days or more, after such administration. In certain embodiments, the methods of the present invention can increase the NITBUT by at least 10% or more, at least 15% or more, beyond the NITBUT measurement obtained the day (or about the time—e.g., ±1 hour) of commencement of the methods of the present invention, which increase, in certain embodiments, can be observed within (or after) about 10 days or more, 20 days or more, or 30 days or more, after administration of the compositions disclosed herein according to the methods of the present invention.

Alternatively, the methods of the present invention can increase the NITBUT in a treated eye of a mammal (i.e., human or rabbit) beyond NITBUT measurement obtained in a nontreated eye of another mammal (i.e., human or rabbit) on the day (or about the time—e.g., ±1 hour) of commencement of the methods of the present invention by up to 1 (or about 1) seconds, up to 1.5 (or about 1.5) seconds or more, or by up to 2 (or about 2) seconds or more, or by up to 3 (or about 3) seconds or more, or by up to 4 (or about 4) seconds or more, after administration, which increase, in certain embodiments, can be observed within (or after) about 10 days or more, 20 days or more, or 30 days or more, after such administration. In these embodiments, the methods of the present invention can increase the NITBUT in a treated eye of a mammal (i.e., human or rabbit) by, at least 10% or more, at least 20% or more, at least 30% or more, at least 40% or more, at least 50% or more, at least 60% or more, at least 70% or more, or at least 80% or more, beyond NITBUT measurements obtained in a nontreated eye of another mammal (i.e., human or rabbit) on the day (or about the time—e.g., ±1 hour) of commencement of the methods of the present invention which increase, in certain embodiments, can be observed within (or after) about 10 days or more, 20 days or more, or 30 days or more after administration of the compositions disclosed herein according to the methods of the present invention.

The ophthalmic compositions may be dispensed to the eye as needed for reducing, relieving or preventing evaporative dry eye symptoms due to meibomian gland disfunction, lacrimal gland dysfunction and/or tear film instability.

When the ophthalmic compositions of the present invention are administered to the eye as eye drops, they are administered at least once per day, at least twice or more, at least three times or more, at least four times or more, at least five or more times, or at least six times or more per day, as needed, to the eye or eyes experiencing tear film instability, meibomian gland dysfunction, or lacrimal dysfunction or signs or symptoms thereof. The intervals between such times of administration can be every 12 hours, every 8 hours, every 6 hours and every 4 hours, as needed. In certain embodiments, reduction in certain of the signs/symptoms of such meibomian gland disfunction, lacrimal gland dysfunction and/or tear film instability may be observed within 1 day, within 2 days, within 3 days, within 4 days, within 5 days, within 6 day, within 7 days, within 8 days, within 9 days, within 10 days, within 15 day, within 20 days, within 25 days, or within 30 days of starting administration of the compositions according to the methods of the present invention.

The compositions used in the methods of the present invention can stored and/or dispensed from multidose containers or from single dose containers. The multidose or single dose containers, in each case, can contain preservatives as described herein or can be free of preservatives.

### EXAMPLES

The compositions used in the methods of the present invention described in following examples illustrate specific embodiments of compositions used in the present invention, but are not intended to be limiting thereof. Other modifications can be undertaken by the skilled artisan without departing from the spirit and scope of this invention.

### Example 1

Table 1 shows a composition in the form of a solution comprising sodium hyaluronate (with lipids/oil) composition suitable for direct application, accordance with the present invention, to the eyes or sounding tissue areas, which composition can be prepared using conventional mixing technology

TABLE 1

| Ingredient | (% w/v) |
|---|---|
| Sodium Hyaluronate | 0.201 |
| Sodium Chloride | 0.055 |
| Potassium Chloride | 0.140 |
| Boric Acid | 0.602 |
| Sodium Borate Decahydrate | 0.050 |
| Polyethylene Glycol 400 | 0.251 |
| Sodium Chlorite | 0.014 |
| Purified Water | QS |

The meibomian gland dysfunction (MGD) rabbit model (Gallois et al, ARVO poster 2021: Development of a meibomian gland dysfunction model in the rabbit—https://iovs.arvojournals.org/article.aspx?articleid=2774732) testing was performed evaluating the composition of Table 1 as follows:
10 New Zealand Rabbits (males, at least 2.5 kg)
   5 day acclimation period
   Group 1: 5 males, Control, Full cautery of meibomian gland in right eye; left eye was not cauterized. No eye drops were administered to group 1 rabbits
   Group 2: 5 males, Test, Full Cautery of Meibomian gland in both left eye and right eye. Eye drops starting Days 15-47, in OS (test composition of Table 1), three times per day
Pre-cauterization steps:
   the test rabbits were sedated with appropriate anesthesia using 35 mg/kg ketamine and 5 mg/kg xylazine (intramuscular) and/or 1-3% Isoflurane (inhalation) prior to the cauterization on Day 0.
   The corneas were topically anaesthetized with 0.5% proparacaine HCl ophthalmic solution.
Cauterization of meibomian gland orifices was performed under a surgical microscope with a standard ophthalmic cautery and care was taken to avoid thermal damage and touching the surrounding tissues. Orifice closure was confirmed (absence of visible meibomian gland orifice/absence of oil on the lid margin and in the tear film, no oil expressed from the glands with digital pressure). The day of cauterization was designated as Day 0. The only the left eye of each rabbit in Group 1 was cauterized and both eyes of each rabbit in Group 2 were cauterized. The rabbits were treated with buprenorphine hydrochloride 0.03 mg/kg twice by subcutaneous injections on the day of cauterization. (Day 0). (Balanced salt solution (BSS) was applied to both eyes to avoid drying of the eyes under anesthesia.)
Post-cauterization steps:
   After the cauterization of meibomian gland orifices, balanced salt solution (BSS) was administered to the surface of the rabbit eyes to keep them moist.
   Animals were treated with buprenorphine hydrochloride 0.03 mg/kg twice by
   subcutaneous injections on the day of cauterization (Day 0).
   The left eye per animal in Group 2 had instilled therein the eye drop of Table 1.
   The composition of Table 1 was instilled in the left eye (OS) three times per day from Day 15 to Day 47.
Test eye application solutions:
   Both eyes per rabbit in Group 1 were left untreated as controls.
   The eyes of the rabbits in Group 2 were treated as follows starting on Day 15 to Day 47:
      30 μL of composition of Table 1 was instilled in the left eye (OS) three times per day (with at least 3 hour intervals between each dose) Clinical observations were conducted pre-study, daily (post treatment-dose) and pre-sacrifice.
Body weights were conducted pre-study and pre-sacrifice.
Draize scores were conducted starting pre-study (Day −2), daily (post treatment-dose) and pre-sacrifice.
Ocular examinations (slit lamp only) were performed Days −1, 7, 14, 15, 19, 22, 29, 36, 43 and 47 (pre-sacrifice). (The performed days had deviation ±2 days if the days are falling into the weekend.)
Fluorescein Staining was performed on Days −1, 14, 22, 29, 36, 43 and 47 (pre-sacrifice). (The performed days had deviation ±2 days if the days are falling into the weekend.)
Non-Invasive Tear Film Breakup Time (NIBUT) in all animals was performed on Days −2, −1, 7, 9, 14 (i.e., on days prior to starting test solution dosing regimen) and on Days 15, 19, 22, 26, 29, 33, 36, 40, 43 and 47 (i.e., on days after commencement of test solution dosing regimen). (The performed days had deviation ±2 days if the days are falling into the weekend.)
   Prior to test dosing regimen, both eyes in animals had NIBUT measurement performed once on Days −2, −1, 7, 9, and 14 prior to test solution dosing administration using a slit lamp (SL-17 Handheld Portable Slit Lamp from Optics Incorporated) with a cobalt blue filter in conjunction with a Tearscope Plus from Keeler. Each eye was blinked three times before the measurement. The eye was placed in proper position for measurement while closed. The Tearscope Plus and the slit lamp were used at the same time to focus on the tear film of the eye and then the time from upstroke of the last blink to the first tear film break (or dry spot formation) was recorded as NIBUT (in seconds). The measurements were performed ±2 days if the scheduled day fell on a weekend or holiday.
   After commencement of the test dosing regimen, both eyes in animals had NIBUT measurement performed three times on Days 15, 19, 22, 26, 29, 33, 36, 40 and 43, and once at termination on day 47 using the slit lamp (SL-17 Handheld Portable Slit Lamp from Optics Incorporated) with a cobalt blue filter in conjunction with a Tearscope Plus from Keeler. NIBUT was performed three times per performed day on each eye: before first test/control solution instillation, after second test solution instillation, and before third test solution instillation. Each eye was blinked three times before the measurement. The eye was placed in proper position while closed. The Tearscope Plus and a slit lamp were used at the same time to focus on the tear film of the eye and then the time from upstroke of the last blink to the first tear film break (or dry spot formation) was recorded as NIBUT (in seconds).
All animals were sacrificed (i.e., euthanized) on Day 48. No gross necropsy was performed. both eyes, superior and inferior eyelids of animals taken out and fixed 10% neutral buffered formalin

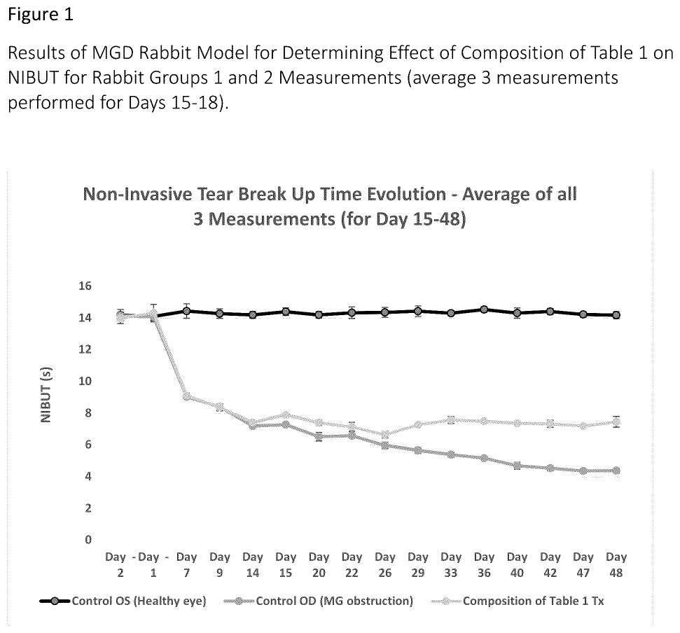

The results of the NIBUT measurements obtained from meibomian gland dysfunction rabbit model are summarized in FIG. 1.

The graph in FIG. 1 illustrates a quantifiable benefit in using the hyaluronic acid solution (Table 1) for improving tear film stability in the model. Specifically, FIG. 1 showed a prevention of the decrease (or worsening or decreasing trend) of tear film breakup time (as observed by the leveling off of the decrease in tear break-up time due to Rabbits' dysfunctional meibomian gland in FIG. 1) after the commencement of the 3 times per day administration of the 30 μL of the 0.2% hyaluronic acid composition of Table 1 as well as a directional increase in NIBUT, namely when considering NIBUTs measured on days 26 to 48; there was an increase of 1.13 seconds which represents a 15% increase in NIBUT over that time frame)) versus the NIBUT as measured on Day 26.

When compared to the average NIBUT of the non-treated, meibomian gland-obstructed eye as measured at the same time point, there was a 2.7 seconds improvement (57% increase) in average NIBUT in the treated eye at Day 40 and 3.1 seconds (70% increase) at Day 48, respectively.

### Embodiments of the Present Invention

1. A method of treating, preventing, relieving or reducing the symptoms associated with tear film deficiency and/or tear film instability in one or both eyes of a subject in need thereof, comprising the step of administering a composition comprising a hyaluronic acid compound to the eye(s), lacrimal gland(s) and/or the meibomian gland(s) of the subject, optionally, which treatment, prevention, relief or reduction is observed within (or after) about 1 day or more, 10 days or more, 20 days or more, or 30 days or more, after such administration.
2. A method of increasing lacrimal secretion in a subject in one or both eyes of need thereof, comprising the step of administering a composition comprising a hyaluronic acid compound to the eye(s) and/or lacrimal gland(s) of the subject, optionally, which increase can be observed within (or after) about 1 day or more, 10 days or more, 20 days or more, or 30 days or more, after such administration.
3. A method of increasing meibomian gland secretion in one or both eyes of a subject in need thereof, comprising the step of administering a composition comprising a hyaluronic acid compound to the eye(s) and/or the meibomian gland(s) of the subject, optionally, which increase can be observed within (or after) 1 day or more, about 10 days or more, 20 days or more, or 30 days or more, after such administration.
4. A method of increasing tear film break up time in one or both eyes of a subject in need thereof, comprising the step of administering a composition comprising a hyaluronic acid compound, lacrimal gland(s) and/or the meibomian gland(s) of the subject, optionally, which increase can be observed within (or after) 1 day or more, about 10 days or more, 20 days or more, or 30 days or more, after such administration.
5. A method of treating, reducing, relieving or preventing low, decreased or decreasing tear film break up times in one or both eyes of a subject in need thereof due to meibomian gland dysfunction, comprising the step of administering a composition comprising a hyaluronic acid compound to the eye(s), lacrimal gland(s) and/or the meibomian gland(s) of the subject, optionally, which treatment, prevention, relief or reduction can be observed within (or after) 1 day or more, about 10 days or more, 20 days or more, or 30 days or more, after such administration.
6. A method of treating, reducing, relieving or preventing evaporative dry eye due to reduced or the inhibition or blockage of meibomian gland secretions in one or both eyes of a subject in need thereof, comprising the step of administering a composition comprising a hyaluronic acid compound to the eye(s), lacrimal gland(s) and/or the meibomian gland(s) of the subject, optionally, which treatment, prevention, relief or reduction can be observed within (or after) about 1 day or more, 10 days or more, 20 days or more, or 30 days or more, after such administration.
7. A method of preventing, reducing, relieving and/or treating meibomian gland and/or lachrymal gland dysfunction in one or both eyes of a subject in need thereof, comprising the step of administering a composition comprising a hyaluronic acid compound to the eye(s), the meibomian gland(s) and/or lachrymal glands of the subject, optionally, which treatment, prevention or reduction can be observed within (or after) about 1 day or more, 10 days or more, 20 days or more, or 30 days or more, after such administration.
8. A method of preventing, reducing, relieving and/or treating evaporative dry eye symptoms due to meibomian gland disfunction, lacrimal gland dysfunction and/or tear film instability in one or both eyes of a subject in need thereof, comprising the step of administering a composition comprising a hyaluronic acid compound to the eye(s), the meibomian gland(s) and/or lachrymal glands of the subject, optionally, which treatment, prevention or reduction can be observed within (or after) about 10 days or more, 20 days or more, or 30 days or more, after such administration.
9. The method of any of the preceding embodiments (or combinations thereof), wherein the step of administering comprises the step of administering an eye drop to the eyes in need thereof.
10. The method of embodiment 9 wherein the eye drop is administered at least once per day, at least twice per day, at least 3 times per day, 3 times per day, at least four times per day, at least five per day, at least six times per day, or as needed to the eyes in need thereof.
11. The method of embodiment 9 wherein the eye drop is administered every 12 hours, every 8 hours, every 6 hours every 4 hours, or as needed.
12. The method of embodiment 9 wherein the eye drops may be selected from multipurpose cleaning, rinsing, disinfecting and storage compositions, rewetting compositions, in-the-eye cleaning compositions.
13. The method of any of the preceding embodiments (or combinations thereof) wherein the step of administering comprises administering a contact lens that has been soaked in the composition prior to administration.
14. The method of embodiment 13 wherein the contact lens is a hydrogel contact lens or a silicone hydrogel contact lens.
15. The method of embodiments 13 or 14 where the contact lens is a daily disposable contact lens.
16. The method of embodiment s 13 or 14 where the contact lens is a reusable contact lens and the contact lens is soaked in a composition used in any of the foregoing claims.
17. The method of any of the preceding embodiments (or combinations thereof) wherein the treatment, prevention, relief or reduction produces an increase in non-invasive tear film break-up time of at least about 10%, about 15% or about 20% compared to the eye without any treatment with the compositions of the present invention.
18. The method of any of the preceding embodiments (or combinations thereof) wherein the treatment, prevention, relief or reduction produces an increase in NITBUT of up to about 1 seconds, at least about 1 second, about 2 seconds, at least about 2 seconds compared to the eye without any treatment with the compositions.
19. The method of any of the preceding embodiments (or combinations thereof) wherein the treatment, prevention, relief or reduction produces an increase in NITBUT of at least 10% or more, at least 15% or more, at least 20% compared to an untreated eye after one day, about 10 days, about 20 days, or about 30 days of administration of the composition.
20. The method of any of the preceding embodiments (or combinations thereof) wherein the treatment, prevention, relief or reduction produces an increase in NITBUT which treatment, prevention or reduction is observed within (or after) about 2 days, 3 days, 4 days, 5 days, 6 day, 7 days, 8 days, 9 days, 15 days, 25 days, of starting administration of the composition.
21. The method of any of the preceding embodiments (or combinations thereof) wherein the treatment, prevention, relief or reduction prevents, treats or reduces tear film deficiencies and contact lens water loss by providing an oil layer at the air-tear interface or additionally at the contact lens-tear interface when a contact lens is present.

What is claimed is:

1. A method of treating, preventing, relieving or reducing the symptoms associated with tear film deficiency and/or tear film instability in one or both eyes of a subject in need thereof, comprising the step of administering a composition comprising a hyaluronic acid compound to the eye(s), lacrimal gland(s) and/or the meibomian gland(s) of the subject, optionally, which treatment, prevention, relief or reduction is observed within (or after) about 1 day or more, 10 days or more, 20 days or more, or 30 days or more, after such administration.

2. A method of increasing lacrimal secretion in a subject in one or both eyes of need thereof, comprising the step of administering a composition comprising a hyaluronic acid compound to the eye(s) and/or lacrimal gland(s) of the subject, optionally, which increase can be observed within (or after) about 1 day or more, 10 days or more, 20 days or more, or 30 days or more, after such administration.

3. A method of increasing meibomian gland secretion in one or both eyes of a subject in need thereof, comprising the step of administering a composition comprising a hyaluronic acid compound to the eye(s) and/or the meibomian gland(s) of the subject, optionally, which increase can be observed within (or after) 1 day or more, about 10 days or more, 20 days or more, or 30 days or more, after such administration.

4. A method of increasing tear film break up time in one or both eyes of a subject in need thereof, comprising the step of administering a composition comprising a hyaluronic acid compound, lacrimal gland(s) and/or the meibomian gland(s) of the subject, optionally, which increase can be observed within (or after) 1 day or more, about 10 days or more, 20 days or more, or 30 days or more, after such administration.

5. A method of treating, reducing, relieving or preventing low, decreased or decreasing tear film break up times in one or both eyes of a subject in need thereof due to meibomian gland dysfunction, comprising the step of administering a composition comprising a hyaluronic acid compound to the eye(s), lacrimal gland(s) and/or the meibomian gland(s) of the subject, optionally, which treatment, prevention, relief or reduction can be observed within (or after) 1 day or more, about 10 days or more, 20 days or more, or 30 days or more, after such administration.

6. A method of treating, reducing, relieving or preventing evaporative dry eye due to reduced or the inhibition or blockage of meibomian gland secretions in one or both eyes of a subject in need thereof, comprising the step of administering a composition comprising a hyaluronic acid compound to the eye(s), lacrimal gland(s) and/or the meibomian gland(s) of the subject, optionally, which treatment, prevention, relief or reduction can be observed within (or after) about 1 day or more, 10 days or more, 20 days or more, or 30 days or more, after such administration.

7. A method of preventing, reducing, relieving and/or treating meibomian gland and/or lachrymal gland dysfunction in one or both eyes of a subject in need thereof, comprising the step of administering a composition comprising a hyaluronic acid compound to the eye(s), the meibomian gland(s) and/or lachrymal glands of the subject, optionally, which treatment, prevention or reduction can be observed within (or after) about 1 day or more, 10 days or more, 20 days or more, or 30 days or more, after such administration.

8. A method of preventing, reducing, relieving and/or treating evaporative dry eye symptoms due to meibomian gland disfunction, lacrimal gland dysfunction and/or tear film instability in one or both eyes of a subject in need thereof, comprising the step of administering a composition comprising a hyaluronic acid compound to the eye(s), the meibomian gland(s) and/or lachrymal glands of the subject, optionally, which treatment, prevention or reduction can be observed within (or after) about 10 days or more, 20 days or more, or 30 days or more, after such administration.

9. The method of claim 1, wherein the step of administering comprises the step of administering an eye drop to the eyes in need thereof.

10. The method of claim 9, wherein the eye drop is administered at least once per day, at least twice per day, at least 3 times per day, 3 times per day, at least four times per day, at least five per day, at least six times per day, or as needed to the eyes in need thereof.

11. The method of claim 9, wherein the eye drop is administered every 12 hours, every 8 hours, every 6 hours every 4 hours, or as needed.

12. The method of claim 9, wherein the eye drops may be selected from multipurpose cleaning, rinsing, disinfecting and storage compositions, rewetting compositions, in-the-eye cleaning compositions.

13. The method of claim 1, wherein the step of administering comprises administering a contact lens that has been soaked in the composition prior to administration.

14. The method of embodiment 13, wherein the contact lens is a hydrogel contact lens or a silicone hydrogel contact lens.

15. The method of embodiments 13 or 14, where the contact lens is a daily disposable contact lens.

16. The method of embodiments 13 or 14, where the contact lens is a reusable contact lens and the contact lens is soaked in a composition used in any of the foregoing claims.

17. The method of any of claim 1, wherein the treatment, prevention, relief or reduction produces an increase in non-invasive tear film break-up time of at least about 10%, about 15% or about 20% compared to the eye without any treatment with the compositions of the present invention.

18. The method of claim 1, wherein the treatment, prevention, relief or reduction produces an increase in NITBUT of up to about 1 seconds, at least about 1 second, about 2 seconds, at least about 2 seconds compared to the eye without any treatment with the compositions.

19. The method of claim 1, wherein the treatment, prevention, relief or reduction produces an increase in NITBUT of at least 10% or more, at least 15% or more, at least 20% compared to an untreated eye after one day, about 10 days, about 20 days, or about 30 days of administration of the composition.

20. The method of claim 1, wherein the treatment, prevention, relief or reduction produces an increase in NITBUT which treatment, prevention or reduction is observed within (or after) about 2 days, 3 days, 4 days, 5 days, 6 day, 7 days, 8 days, 9 days, 15 days, 25 days of starting administration of the composition.

21. The method of claim 1, wherein the treatment, prevention, relief or reduction prevents, treats or reduces tear film deficiencies and contact lens water loss by providing an oil layer at the air-tear interface or additionally at the contact lens-tear interface when a contact lens is present.
